(19) United States
(12) Patent Application Publication
(10) Pub. No.: US 2015/0182489 A1
(43) Pub. Date: Jul. 2, 2015

(54) GOLD (III) COMPOUNDS AND USE FOR TREATING CANCER

(71) Applicants: King Abdulaziz City for Science and Technology, Riyadh (SA); King Fahd University of Petroleum and Minerals, Dhahran (SA)

(72) Inventors: Ayesha AHMED, AI-Khobar (SA); Dalal Mohammed AL-TAMIMI, AI-Khobar (SA); Anvarhusein Abdulkadir ISAB, Dhahran (SA); Abdulaziz Mansour ALKHAWAJAH, Dammam (SA); Mohamed Abdel-Aala EL-SHAWARBY, AI-Khobar (SA)

(73) Assignees: King Abdulaziz City for Science and Technology, Riyadh (SA); King Fahd University of Petroleum and Minerals, Dhahran (SA)

(21) Appl. No.: 14/142,328

(22) Filed: Dec. 27, 2013

(51) Int. Cl. A61K 31/28

(52) U.S. Cl. CPC A61K 31/28

(57) ABSTRACT

Metallodrugs including gold (III) compound with various polydentate ligands, the renal and hepatic toxicity of gold (III) compounds with various polydentate ligands, an antineoplstic drug containing the gold (III) compound [Au(en)Cl₂]Cl and a process of administering the compound [Au(en)Cl₂]Cl to a patient in need of treatment for cancer.

### BACKGROUND

1. Field of the Disclosure

The invention relates to metallo-drugs for cancer treatment including gold (III) compounds, methods of making gold (III) compounds and methods of treating cancer by administering gold (III) compounds.

2. Description of the Related Art

Gold is a noble metal and a commonly used material due to its oxidation resistance and unique electrical, magnetic, optical and physical characteristics. It exists in multiple oxidation states ranging from −1 to +5; the predominant form being Au (I) and Au (III). Metallic gold is known to be an inert and nontoxic metal. It is only the gold salts and radioisotopes that have pharmacological significance (see for example Nagender et al., “Gold and nano-gold in medicine: overview, toxicology and perspectives”, J. Appl. Biomed. 7(2): 75-91 (2009)—incorporated herein by reference in its entirety).

The use of gold compounds as medicinal agents is referred to as chrysotherapy (Pacheco et al., “Biomedical Applications of Gold and Gold Compounds,” in: Cort, C. and Holliday, R. (Editors), Gold Science and Applications, World Gold Council, London, CRC Press pp. 217-230 (2009)—incorporated herein by reference in its entirety). Medical and therapeutic use of gold dates back to thousands of years (Milacic et al., “Gold complexes as prospective metal based anticancer drugs,” Histol. Histopathol. 23(1): 101-8 (2008)). In ancient cultures, around 2500 BC, gold was considered an integral component in the treatment of diseases such as measles, skin ulcers, and smallpox (Kean et al., “The history of gold therapy in rheumatoid disease”, Semin. Arthritis Rheum., 14(3): 180-6. (1985) and Mandihassan, “Cinnabar-gold as the best alchemical drug of longevity called Makaradhwaja in India,” Am. J. Chin. Med., 13(1-4): 93-108 (1985)—both incorporated herein by reference in their entireties).

In the 16th century, gold was recommended for the treatment of epilepsy. Its rational medicinal use began in the early 1920's when it was introduced as a treatment of tuberculosis (Daniel et al., “Gold Nanoparticles: assembly, supramolecular chemistry, quantum-size related properties, and applications toward biology, catalysis, and nanotechnology,” Chem. Rev. 104(1): 293-346 (2004)—incorporated herein by reference in its entirety). Gold as an anti rheumatic agent was first reported in 1929 (Kean et al., “Gold therapy II. Historical, chemical, pharmacological and biological profile of anti-arthritic gold compounds,” Singapore Med J 28(2): 117-25 (1987)—incorporated herein by reference in its entirety). Gold and gold compounds are now mostly used for the treatment of various diseases including psoriasis, palindromic rheumatism, juvenile arthritis and discoid lupus erythematosus (Felson et al., “The comparative efficacy and toxicity of second-line drugs in rheumatoid arthritis: Results of two meta analyses”, Arthritis. Rheum. 33(10): 1449-61 (1990) and Shaw, “Gold-based therapeutic agents,” Chem. Rev. 99(9): 2589-2600 (1999)—both incorporated herein by reference in their entireties). However, following the body's extensive exposure to gold compounds, it can diffuse to various organs like liver, kidney and spleen. Skin irritation, mouth ulcers, nephrotoxicity, liver toxicity and blood disorders have been associated with prolonged exposure to gold compounds (Bhattacharya et al., “Biological properties of “naked” metal nanoparticles”, Adv Drug Deliv Rev 60: 1289-1306 (2008)—incorporated herein by reference in its entirety).

Currently gold complexes have gained considerable attention due to their strong antiproliferative (Wu et al., “Cholestatic hepatitis caused by acute gold potassium cyanide poisoning,” Clin. Toxicol. 39: 739-43 (2001); Cattaruzza et al., “Antitumor activity of gold(III)-dithiocarbamato derivatives on prostate cancer cells and xenografts,” Int. J. Cancer 128(1): 206-15 (2011); Rosenberg et al., “Inhibition of Cell Division in Escherichia coli by Electrolysis Products from a Platinum Electrode”, Nature 205: 698-9 (1965); Rosenberg et al., “Platinum compounds: a new class of potent antitumour agents”, Nature 222(5191): 385-6 (1969)—each incorporated herein by reference in their entireties) and antiangiogenic potential. The spectrum of gold complexes with documented cell growth inhibiting properties include a large variety of different ligands attached to gold in the oxidation states+1 or +3, that is gold (I) and gold (III) compounds (Galanski et al., “Update of the Preclinical Situation of Anticancer Platinum Complexes: Novel Design Strategies and Innovative Analytical Approaches”, Curr. Med. Chem. 12(18): 2075-94 (2005) and Ott, “Review On the medicinal chemistry of gold complexes as anticancer drugs”, Coord. Chem. Rev. 253(11-12): 1670-81 (2009)—each incorporated herein by reference in their entireties). Gold (I) complexes proved to be unsuitable for clinical practice due to accompanying cardiotoxicity (Schmidbauer H., “Gold-progress in chemistry, biochemistry and technology,” Chichester, John Wiley & Sons (1999) and Hoke et al., “In vivo and in vitro cardiotoxicity of a gold containing antineoplastic drug candidate in the rabbit, “Toxicol. Appl. Pharmacol. 100(2): 293-306GD (1989)—each incorporated herein by reference in their entireties), while studies on gold (III) complexes are comparatively scarce. Gold (III) bears homology to cisplatin as it is isoelectronic with platinum (II) and tetracoordinate gold (III) complexes have the same square-planar geometries as cisplatin.

Cisplatin [i.e., cis-diamminedichloroplatinum(II)] is one of the most widely employed drugs in cancer chemotherapy, discovered more than 40 years ago, and it became the first FDA-approved platinum anticancer compound in 1978 (Tiekink, “Gold derivatives for the treatment of cancer”, Crit. Rev. Oncol. Hematol. 42: 225-48 (2002)—each incorporated herein by reference in their entireties). Its effectiveness in solid tumoral lesions is markedly hampered by severe toxic side effects comprising predominantly nephrotoxicity (Kelland L., “The resurgence of platinum-based cancer chemotherapy,” Nat. Rev. Cancer 7: 573-84 (2007) and Meijer et al., “Nephrotoxicity of cis-diamminedichloride platinum [CDDP] during remissioninduction and maintenance chemotherapy of the testicular carcinoma,” Cancer Chemother. Pharmacol. 8: 27-30 (1982)—each incorporated herein by reference in their entireties), development of tumor resistance (Brock et al., “Partial reversibility of cisplatin nephrotoxicity in children,” J. Pediatr. 118: 531-4.23 (1991); Chao et al., “An integrative approach to identifying cancer chemoresistance-associated pathways,” BMC Medical Genomics 4(1): 23-37 (2011); Yamashita et al., “The Role of PARP 1 for Cisplatin-Based Chemoresistance,” Otolaryngol. Head Neck Surg. 143(2): 54-60 (2010); Oliver et al., “Chronic cisplatin treatment promotes enhanced damage repair and tumor progression in a mouse model of lung cancer”, Genes & Dev. 24: 837-852 (2010)—each incorporated herein by reference in their entireties) and occurrence of secondary malignancies that contributes a high treatment failure ratio in clinical management.

Current studies aim towards designing newer compounds showing enhanced anti-proliferative potential and less associated toxicity than cisplatin. In this regards, gold (III) complexes with various ligands like Au—N, Au—S or Au—C bonds are being extensively investigated for their bioactivities as antiproliferative agents (Ott et al., “Non Platinum Metal Complexes as Anti-cancer Drugs,” Arch. Pharm. Chem. Life Sci. 340: 117-126 (2007)—incorporated herein by reference in its entireties) and simultaneously new combinations of complexes are being developed. Milovanovic et al. concluded that gold (III) complexes are much faster to react with nucleophiles in comparison to Pt(II) complexes. Milovanovic also demonstrated that gold (III) complexes exhibit relevant cytotoxic properties when tested on chronic lymphocytic leukemia cells (CLL). Mehboob et al. described the properties and synthesis of gold (III) alkanediamine complexes (Mehboob et al., “Synthesis, spectroscopic characterization and anti-cancer properties of new gold (III)—alkanediamine complexes against gastric, prostate and ovarian cancer cells; crystal structure of [Au₂(pn)₂(Cl)₂]Cl₂—H₂O”, Polyhedron 61 (2013) 225-234—incorporated herein by reference in its entirety).

### SUMMARY

In order to address the drawbacks, especially cytotoxicity and reactivity, gold (III) compounds were developed and tested as cancer treatment drugs.

An embodiment of the invention includes a monomeric gold (III) compound of a gold cation comprising a single bidentate ligand chelated to a gold atom through two nitrogen atoms and bonded to two halide atoms and having an anionic counter ion.

In another embodiment the invention includes a gold (III) compound that having one or more polydentate ligands.

In another embodiment of the invention the gold (III) compound includes at least one bidentate ligand and the gold (III) is in the form of an anion.

In another embodiment of the invention the gold (III) compound includes a single bidentate diamine ligand.

In another embodiment the invention includes a gold (III) compound of formula [Au(en)Cl₂]Cl.

A further embodiment of the invention is a process for making gold (III) compounds containing one or more polydentate ligands.

In another embodiment the invention includes an anti-neoplastic drug comprising the gold (III) compound and one or more pharmaceutically acceptable materials.

In another embodiment the invention includes a method of treating a patient in need of treatment for cancer.

In another embodiment the invention includes a method of administering a cytotoxically effective amount of the gold (III) compound to a patient.

In another embodiment the invention includes a method of administering a cytotoxically effective amount of the gold (III) compound to a patient in an amount such that no renal tubular necrosis occurs in the patient.

In another embodiment the invention includes a method of administering a cytotoxically effective amount of the gold (III) compound to a patient in an amount to produce less hepatic toxicity in comparison to the administration of a cis-platin drug.

In another embodiment of the invention a gold (III) compound causes low histological changes in kidney and liver when administered to a patient in need of treatment of cancer.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a spectrum of renal tubular necrosis in acute toxicity study of [Au(en)Cl₂]Cl;

FIG. 2A shows microscopic findings of renal tubules showing different grades of renal tubular necrosis in a study of the gold (III) compound [Au(en)Cl₂]Cl at an H&E ×20 magnification;

FIG. 2B shows microscopic findings of renal tubules showing different grades of renal tubular necrosis in a study of the gold (III) compound [Au(en)Cl₂]Cl at an H&E ×40;

FIG. 2C shows microscopic findings of renal tubules showing different grades of renal tubular necrosis in a study of the gold (III) compound [Au(en)Cl₂]Cl Grade 1 in H&E ×40 magnification;

FIG. 2D shows microscopic findings of renal tubules showing different grades of renal tubular necrosis in a study of the gold (III) compound [Au(en)Cl₂]Cl Grade 1 in H&E ×4 magnification;

FIG. 2E shows microscopic findings of renal tubules showing different grades of renal tubular necrosis in a study of the gold (III) compound [Au(en)Cl₂]Cl Grade 5 in H&E ×20 magnification;

FIG. 2F shows microscopic findings of renal tubules showing different grades of renal tubular necrosis in a study of the gold (III) compound [Au(en)Cl₂]Cl Grade 5 in H&E ×40 magnification;

FIG. 3A shows renal hepatic tissues in acute toxicity showing mild congestion and no pathological change in H&E ×40 magnification;

FIG. 3B shows renal hepatic tissues with hepatic tissue in acute toxicity with mild congestion in H&E ×40 magnification;

FIG. 3C shows renal hepatic tissues with marked ballooning degeneration in H&E ×40 magnification;

FIG. 3D shows renal hepatic tissues with unremarkable renal tubules in sub-acute toxicity controls with H&E ×40 magnification;

FIG. 3E shows renal hepatic tissues unremarkable hepatic tissue in sub-acute toxicity controls with H&E ×20 magnification;

FIG. 3F shows renal hepatic tissues unremarkable hepatic tissue in sub-acute toxicity controls with H&E ×40 magnification;

FIG. 4 shows the extent of hepatic steatosis in an acute toxicity study of a gold (III) compound;

FIG. 5A shows a spectrum of hepatic microscopic findings in an acute toxicity study of a gold (III) compound with marked mixed micro and macro vesicular steatosis at H&E ×40 magnification;

FIG. 5B shows a spectrum of hepatic microscopic findings in an acute toxicity study of a gold (III) compound with marked sinusoidal congestion and dilation at H&E ×20 magnification;

FIG. 5C shows a spectrum of hepatic microscopic findings in an acute toxicity study of a gold (III) compound with marked sinusoidal congestion and dilation at H&E ×40 magnification;

FIG. 5D shows a spectrum of hepatic microscopic findings in an acute toxicity study of a gold (III) compound marked ballooning degeneration with microgranulomas at H&E ×40 magnification;

FIG. 6A shows renal tubules having no evidence of necrosis in sub-acute toxicity studies of a gold (III) compound at a H&E ×10 magnification;

FIG. 6B shows renal tubules having no evidence of necrosis in sub-acute toxicity studies of a gold (III) compound at an H&E ×20 magnification;

FIG. 6C shows renal tubules having no evidence of necrosis in sub-acute toxicity studies of a gold (III) compound at an H&E ×40 magnification;

FIG. 7A shows hepatic microscopic findings in sub-acute toxicity studies of a gold (III) compound with mild ballooning at an H&E ×20 magnification;

FIG. 7B shows hepatic microscopic findings in sub-acute toxicity studies of a gold (III) compound with mild ballooning degeneration at an H&E ×20 magnification;

FIG. 7C shows hepatic microscopic findings in sub-acute toxicity studies of a gold (III) compound with marked ballooning degeneration at an H&E ×20 magnification;

FIG. 7D shows marked ballooning degeneration at an H&E ×40 magnification.

### DETAILED DESCRIPTION OF THE EMBODIMENTS

Referring now to the drawings, wherein like reference numerals designate identical or corresponding parts throughout the several views.

One aspect of the invention includes a gold (III) compound, preferably a compound of formula [Au(L)X₂)]X′ where L is a polydentate Lewis acid that coordinates, preferably chelates, the gold atom, X is a halogen atom and X′ is a counter ion. The [Au(L)X₂)] core of the gold (III) compound is cationic and carries a single positive charge. The counter ion X′ is an anion and may thus likewise represent a halogen atom. The anion X′ may also be other anions such as PF₆⁻ and the like. X′ may be any pharmaceutically acceptable anion including, as noted above, halide, hexafluorophosphate, nitrate, and triflate. It is preferred that the anion X′ is Cl⁻.

The ligand L is a bidentate or polydentate Lewis base which functions to donate at least two lone pairs of electrons to the gold atom at the center of the gold (III) compound. The ligand is preferably uncharged and neutral but may include cationic or anionic bidentate materials such as acetylacetonate (acac). Polyanions such as oxalate ions may also function as the ligand of the gold (III) compound.

Other ligands that act as chelating agents include anionic biodegradable chelating agents; carbonates, such as sodium and potassium carbonate; citric acid; dicarboxymethylglutamic acid; aminopolycarboxylic acid type chelating agents, including but not limited to cyclohexylenediamintetraacetic acid (CDTA), diethylenetriamine-pentaacetic acid(DTPA), ethylenediaminedisuccinic acid (EDDS); ethylenediaminetetraacetic acid (EDTA), hydroxyethylethylenediaminetriacetic acid (HEDTA), hydroxyethyliminodiacetic acid (HEIDA), nitrilotriacetic acid (NTA), and the sesquisodium salt of diethylene triamine penta (methylene phosphonic acid), or mixtures thereof; inulins (e.g. sodium carboxymethyl inulin); malic acid; nonpolar amino acids, such as methionine and the like; oxalic acid; phosphoric acids; phosphonates, in particular organic phosphonates such as sodium aminotrismethylenephosphonate; phosphonic acids and their salts, including but not limited to ATMP (aminotri-(methylenephosphonic acid)), HEDP (1-hydroxyethylidene-1,1-phosphonic acid), HDTMPA (hexamethylenediaminetetra-(methylenephosphonic acid)), DTPMPA (diethylenediaminepenta-(methylenephosphonic acid)), and 2-phosphonobutane-1,2,4-tricarboxylic acid; phosphate esters; polyaminocarboxylic acids; polyacrylamines; polycarboxylic acids; polysulphonic acids; phosphate esters; inorganic phosphates; polyacrylic acids; phytic acid and derivatives thereof (especially carboxylic derivatives); polyaspartates; polyacrylades; polar amino acids (both alph- and beta-form), including but not limited to arginine, asparagine, aspartic acid, glutamic acid, glutamine, lysine, and ornithine; succinic acid; trihydroxamic acid and derivatives thereof, as well as combinations of the above-listed chelating agents, and the free acids of such chelating agents (as appropriate) and their water-soluble salts (e.g., their Na⁺, K⁺, NH₄⁺, and Ca²⁺ salts).

Non-limiting exemplary chelating agent/metal complexes which may be formed by the chelating agents of the present disclosure with suitable metal ions include chelates of the salts of barium (II), calcium (II), strontium (II), magnesium (II), chromium (II), titanium (IV), aluminum (III), iron (II), iron (III), zinc (II), nickel (II), tin (II), or tin (IV) as the metal and nitrilotriacetic acid, 1,2-cylohexane-diamine-N,N,N′,N′-tetra-acetic acid, diethylenetriamine-pentaacetic acid, ethylenedioxy-bis(ethylene-nitrilo)-tetraacetic acid, N-(2-hydroxyethyl)-ethylenediamino-N,N′,N-triacetic acid, triethylene-tetraamine-hexaacetic acid or N-(hydroxyethyl)ethylenediamine-triacetic acid or a mixture thereof as a ligand

Preferred neutral or mono- or dianionic bidentate or polydentate ligands are selected from diamines, such as, for example, ethylenediamine, N,N,N′,N′-tetramethylethylenediamine, propylenediamine, N,N,N′,N′-tetramethylpropylenediamine, cis- or trans-diaminocyclohexane, cis- or trans-N,N,N′,N′-tetramethyldiaminocyclohexane, imines, such as, for example, 2-[1-(phenylimino)ethyl]pyridine, 2[1-(2-methylphenylimino)ethyl]pyridine, 2[1-(2,6-di-iso-propylphenylimino)ethyl]pyridine, 2-[1-(methylimino)ethyl]-pyridine, 2-[1-(ethylimino)ethyl]pyridine, 2[1-(iso-propylimino)ethyl]pyridine, 2[1-(tert-butylimino)ethyl]pyridine, diimines, such as, for example, 1,2-bis(methylimino)ethane, 1,2-bis(ethylimino)ethane, 1,2-bis(iso-propylimino)ethane, 1,2-bis(tert-butylimino)ethane, 2,3-bis(methylimino)butane, 2,3-bis(ethylimino)butane, 2,3-bis(iso-propylimino)butane, 2,3-bis(tert-butylimino)butane, 1,2-bis(phenylimino)ethane, 1,2-bis(2-methylphenylimino)ethane, 1,2-bis(2,6-di-iso-propylphenylimino)ethane, 1,2-bis(2,6-di-tert-butylphenylimino)ethane, 2,3-bis(phenylimino)butane, 2,3-bis(2-methylphenylimino)butane, 2,3-bis(2,6-di-iso-propylphenylimino)butane, 2,3-bis(2,6-di-tertbutylphenylimino)butane, heterocycles containing two nitrogen atoms, such as, for example, 2,2′-bipyridine, o-phenanthroline, diphosphines, such as, for example, bis(diphenylphosphino)methane, bis(diphenylphosphino)ethane, bis(diphenylphosphino)propane, bis(diphenylphosphino)butane, bis(dimethylphosphino)methane, bis(dimethylphosphino)ethane, bis(dimethylphosphino)propane, bis(diethylphosphino)methane, bis(diethylphosphino)ethane, bis(diethylphosphino)propane, bis(di-tert-butylphosphino)methane, bis(di-tert-butylphosphino)ethane, bis(tert-butylphosphino)propane, 1,3-diketonates derived from 1,3-diketones, such as, for example, acetylacetone, benzoylacetone, 1,5-diphenylacetylacetone, dibenzoylmethane, bis(1,1,1-trifluoroacetyl)methane, 3-ketonates derived from 3-ketoesters, such as, for example, ethyl acetoacetate, carboxylates derived from aminocarboxylic acids, such as, for example, pyridine-2-carboxylic acid, quinoline-2-carboxylic acid, glycine, N,N-dimethylglycine, alanine, N,N-dimethylaminoalanine, salicyliminates derived from salicylimines, such as, for example, methylsalicylimine, ethylsalicylimine, phenylsalicylimine, dialcoholates derived from dialcohols, such as, for example, ethylene glycol, 1,3-propylene glycol, and dithiolates derived from dithiols, such as, for example, 1,2-ethylenedithiol, 1,3-propylenedithiol.

Bidentate monoanionic ligands may include a cyclometallated five-membered ring or six-membered ring with the gold atom (e.g., complexes having at least one metal-carbon bond), in particular a cyclometallated five-membered ring. These are, in particular, ligands such as the phenylpyridine, naphthylpyridine, phenylquinoline, phenylisoquinoline, etc., type, each of which may be substituted by one or more radicals R. In other embodiments of the invention a tridentate or higher polydentate ligand is used but the ligand bonds to the gold metal with only two of a plurality of available pie-bonding orbitals. In such a configuration the polydentate ligand acts as a bidentate ligand even though additional Lewis acid sites are available for binding. In a still further aspect of the invention the counter ion to the gold (III) compound itself has one or more ligands which may separate bind a different gold atom of a second gold (III) compound which is the same or different from the gold (III) compound to which the counter ion serves as a balancing charge.

The gold (III) compound is preferably in the form of a dichlorido(ethylenediamine)-aurate (III) ion. This gold (III) compound is shown below together with a chloride counter ion as formula (Ia):

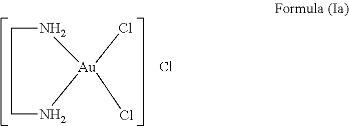

In an embodiment, the antineoplastic drugs containing the gold (III) compound of the present invention may require different routes of administration, because of their different physical and chemical characteristics. For example, the gold (III) compound may be administered either orally or parenterally to generate and maintain good blood levels thereof, while the antineoplastic agent may be administered parenterally, by intravenous, subcutaneous or intramuscular route.

The antineoplastic drugs containing the gold (III) compound may be administered locally. Routes for local administration in general include, for example, topical administration routes but also intravesical, intradermal, transdermal, subcutaneous, or intramuscular injections or intralesional, intracranial, intrapulmonal, intracardial, and sublingual injections. More preferably, the inventive pharmaceutical composition may be administered by an intravesical route. The suitable amount of the inventive pharmaceutical composition to be administered can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof.

For oral use, the gold (III) compound may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc and sugar.

For intramuscular, intraperitoneal, subcutaneous and intravenous use, sterile solutions of the gold (III) compound are usually employed, and the pH of the solutions should be suitably adjusted and buffered. According to a further specific embodiment of the present invention, the inventive pharmaceutical composition as defined herein typically comprises an pH-value of about 3 to about 8, preferably of about 3 to about 7, more preferably of about 3 to about 6, even more preferably of about 3 to about 5, and most preferably a pH-value of about 3.5 to about 4, including a pH-value in a range of about 3.5 to about 4.9, of about 3.5 to about 4.8, of about 3.6 to about 4.7, of about 3.6 to about 4.6, of about 3.7 to about 4.5, of about 3.7 to about 4.4, of about 3.8 to about 4.3, of about 3.8 to about 4.2, or of about 3.9 to about 4.1.

The inventive pharmaceutical composition may be prepared and administered in a pH-value as defined above. If necessary, the pH-value may be further adjusted for the specific treatment and administration requirements, e.g. to a more neutral pH-value of about 5, 6, or 7 (pH 5 to 7), e.g. using buffers and additives as disclosed herein.

In an embodiment, the sterile solutions of the active ingredient used are prepared in saline or distilled water. The actual dosage of the active ingredients i.e. the anticancer agents contained in the combination may be varied depending upon the requirements of the patient and the severity of the condition being treated. Generally, treatment is initiated with smaller doses, which are less than the optimum dose of the compound. Thereafter, the dose of each ingredient is increased by small amounts until the optimum effect under the circumstances is reached. However, the amount of each ingredient in the pharmaceutical combination will typically be less than an amount that would produce a therapeutic effect if administered alone. For convenience, the total daily dose may be divided and administered in portions during the day if desired. In an embodiment, paclitaxel or its pharmaceutically acceptable salt, and the antineoplatic drug containing the gold (III) compound are administered sequentially in injectable forms, such that paclitaxel is administered in a synergistically effective dose ranging from 10 mg to 1000 mg each, and the CDK inhibitor is administered in a synergistically effective dose ranging from 5 mg/m²/day to 1000 mg/m²/day, particularly in a dose ranging from 9 mg/m²/day to about 259 mg/m²/day.

In an embodiment, the antineoplastic drug containing the gold (III) compound is provided for use in the treatment of a cancer is administered to a subject in need thereof, for six to eight treatment cycles, particularly six treatment cycles; two consecutive treatment cycles comprising the following steps: i) a single dose administration of the antineoplastic drug containing the gold (III) compound on day one of the treatment cycle; ii) from second day, administration of one dose per day of antineoplastic drug containing the gold (III) compound for four consecutive days; iii) a two-day interval wherein no drug (antineoplastic agent) is administered; iv) optional administration of antineoplastic drug containing the gold (III) compound for five consecutive days followed by two-day interval with no drug (antineoplastic agent) administration; v) optionally repeating step iv); and vi) repeating steps i) to v) as a second treatment cycle, after an interval of three weeks from the beginning of step i).

In an embodiment, the antineoplastic drugs containing the gold (III) compound is administered to a subject in need thereof, for two to six treatment cycles, before surgery or after surgery or partially before and partially after surgery.

The inventive antineoplastic drugs containing the gold (III) compound typically comprises a “safe and effective amount” of the components of the inventive the antineoplastic drugs containing the gold (III) compound, particularly of the gold (III) compound. As used herein, a “safe and effective amount” means an amount of these component, particularly of the gold (III) compound and derivatives thereof, that is sufficient to significantly induce a positive modification of a disease or disorder as defined herein. At the same time, however, a “safe and effective amount” is small enough to avoid serious side-effects, that is to say to permit a sensible relationship between advantage and risk. The determination of these limits typically lies within the scope of sensible medical judgment. A “safe and effective amount” of the components of the antineoplastic drug containing the gold (III) compound will furthermore vary in connection with the particular condition to be treated and also with the age and physical condition of the patient to be treated, the body weight, general health, sex, diet, time of administration, rate of excretion, drug combination, their activity, the severity of the condition, the duration of the treatment, the nature of the accompanying therapy, of the particular pharmaceutically acceptable carrier used, and similar factors, within the knowledge and experience of the accompanying doctor. The inventive pharmaceutical composition may be used for human and also for veterinary medical purposes, preferably for human medical purposes.

Without being bound thereto, in some embodiments, the inventive antineoplastic drugs containing the gold (III) compound will contain or release sufficient active gold (III) compound to provide a dose of about 10, 20, 50, or 100 nanograms per kilogram (ng/kg) to about 50 milligrams per kilogram (mg/kg), preferably about 10 micrograms per kilogram (.mu.g/kg) to about 5 mg/kg, of the compound or a salt thereof to the subject. In other embodiments, the inventive antineoplastic drugs containing the gold (III) compound will contain or release sufficient active gold (III) compound to provide a dose of, for example, from about 0.0001, 0.001, 0.01 or 0.01 mg/m² to about 5.0 mg/m², computed according to the Dubois method, in which the body surface area of a subject (m²) is computed using the subject's body weight: m²=(wt kg^{0.425} times height cm 0.725) times 0.007184, although in some embodiments the methods may be performed by administering a compound or salt or composition in a dose outside this range. In some of these embodiments, the method includes administering sufficient imidazochinolin(amine) or a derivative thereof to provide a dose of from about 0.0001, 0.001, 0.01, or 0.1 mg/m² to about 2.0 mg/m² to the subject, for example, a dose of from about 0.004, 0.04, or 0.4 m g/m² to about 1.2 mg/m².

In another aspect of the present invention, it is provided a composition which comprises the gold (III) compound in an amount effective for mitigating tissue damage or lethality induced by an agent. In some embodiments, the composition includes a compound in an effective amount for a condition selected from conditions related to radiation-induced lethality, conditions related to radiation-induced genotoxicity and cytotoxicity, conditions related to radiation-induced damage to healthy tissues during radiation therapy, conditions related to radiation-induced persistent genetic instability, conditions related to ultraviolet (UV) radiation-induced damage, conditions related to damage induced by chemical carcinogens, radiation-induced cancer, spontaneous cancer, or aging.

In some embodiments of the composition, the antineoplastic drugs containing the gold (III) compound and/or the composition can further optionally include at least one other therapeutic agent. In some embodiments the antineoplastic drug containing the gold (III) compound and/or the composition further comprises an excipient and/or a pharmaceutically acceptable carrier.

The gold (III) compounds of the present invention have antineoplastic activity and can therefore be used for the treatment or prevention of tumors, in particular solid tumors, such as astrocytoma, bladder cancer, bone cancer, brain tumor, breast cancer, bronchial tumor, cervical cancer, colorectal cancer, endometrial cancer, esophageal cancer, gallbladder cancer, gastrointestinal stromal tumor, germ cell tumor, glioma, head and neck cancer, liver cancer, lymphoma, sarcoma, lung cancer, melanoma, ovarian cancer, pancreatic cancer, thyroid cancer, neuroblastoma, prostate cancer, renal cancer, skin cancer, squamous neck cancer, stomach (gastric) cancer, testicular cancer. The compounds of the invention are especially useful for treatment or prevention of cervical cancer, colorectal cancer, gastrointestinal stromal tumor, liver cancer, lung cancer, ovarian cancer, prostate cancer, stomach cancer, and pancreatic carcinoma.

The gold (III) compound may be used in an antineoplastic drug in combination with one or more other antineoplastic agents. Examples for antineoplastic agents are aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosfamide, irinotecan, lomustine, mechlorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cis-platin, carboplatin, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguanine), tipifarnib. Examples for antineoplastic agents which are protein kinase inhibitors include imatinib, erlotinib, sorafenib, sunitinib, dasatinib, nilotinib, lapatinib, gefitinib, temsirolimus, everolimus, rapamycine, bosutinib, pzopanib, axitinib, neratinib, vatalanib, pazopanib, midostaurin and enzastaurin. Examples for antineoplastic agents which are antibodies comprise trastuzumab, cetuximab, panitumumab, rituximab, bevacizumab, mapatumumab, conatumumab, lexatumumab and the like.

### Examples

The gold (III) compound [Au(en)Cl₂]Cl wherein (en) is an N-substituted ethylenediamine was prepared according to Isab, A. A., et al., Acta, Part A 79 (2011) 1196 and fully characterized by spectroscopic techniques such as UV-Vis, Far-IR, IR spectroscopy, solution, X-ray and solid NMR. The solution NMR was measured in D₂O, implicating that it is water soluble (Al-Maythalony et al., “Synthesis and characterization of gold(III) complexes with alkyldiamine ligands”, Inorg. Chim. Acta. 362: 3109-13 (2009) and Zhu et al., “Synthesis, Structures, and Electrochemistry of Gold(III) Ethylenediamine Complexes and Interactions with Guanosine 5′-Monophosphate,” Inorg. Chem. 45 (6): 2688-94 (2006)—each incorporated herein by reference in their entirety). In the current study we evaluated the histopathological toxicity of this compound in renal and hepatic tissues of rats.

Materials and Methods

Study of the gold (III) compound [Au(en)Cl₂]Cl was carried out in Pathology Department, College of Medicine, University of Dammam. It was compartmentalized into two segments comprising acute toxicity and subacute toxicity studies. For both segments, Albino Wistar male rats (n=42), weighing 200-250 gram were obtained from the College of Veterinary Medicine, King Faisal University, Al-Hassa, Saudi Arabia. They were placed in an animal house under standardized conditions, fed standard chow and exposed to an optimized environment one week before the start of the experiment.

Acute Toxicity Study

In acute toxicity, 5 groups of rats (A/I-E/I), with each group comprising 5 animals, were administered gold compound intraperitoneally in doses of 1500 mg/kg, 750 mg/kg, 375 mg/kg, 187.5 mg/kg and 93.75 mg/kg, respectively. A control group of 5 animals (F/I) was simultaneously administered 0.2 ml water intraperitoneally.

After 24 hours, the number of deceased rats was counted in each group and LD50 (dose that kills 50% of animals) was calculated (322 mg/kg) by the method of Miller and Tainter (Miller et al., “Estimation of LD50 or ED50 values and their errors using Log-Probit graph paper,” Proc. Soc. Expt. Biol. Med., 57: 261-264 (1937)—incorporated herein by reference in its entireties).

Autopsy was carried out in all animals and renal as well as hepatic tissues were preserved in 10% buffered formalin for subsequent evaluation of histopathological alterations.

Sub-Acute Toxicity Study

The rats in this component of the study were divided into two treatment groups, A/II and B/II, with six rats in each. Group “A/II” served as the experimental group while group “B/II” served as the control. Rats in the experimental group (A/II) were injected with 32.2 mg/kg ( 1/10 of LD50) body weight of the gold compound while rats in the control group (B/II) were injected with normal saline daily for 14 days.

Autopsy was carried out in all the rats. Renal and hepatic tissues were preserved in 10% buffered formalin until subjected to histopathological evaluation.

Histopathological Work Up

Fixation and tissue processing. The formalin preserved hepatic and renal tissue samples of [Au(en)Cl₂]Cl dosed rats and controls were processed in an automated tissue processor (Tissue-tek VIP-5, from SAKURA). The processing consisted of an initial 2 step fixation comprising tissue immersion in 10% buffered formalin for two hours each, followed by removal of fixative in distilled water for 30 minutes. Dehydration was then carried out by running the tissues through a graded series of alcohol (70%, 90%, and 100%). The tissue was initially exposed to 70% alcohol for 30 minutes followed by 90% alcohol for 1 hour and then two cycles of absolute alcohol, each for one hour.

Dehydration was then followed by clearing the samples in several changes of xylene. It consisted of tissue immersion for an hour in a mixture comprising 50% alcohol and 50% xylene, followed by pure xylene for one and a half hour. Samples were then impregnated with molten paraffin wax, then embedded and blocked out. Paraffin sections (4-5 um) were stained with hematoxylin and eosin, the conventional staining technic (Underwood, J C E., “Histochemistry. Theoretical and applied. Vol. 2: Analytical technology Pearse AGE,” Fourth edition, Churchill Livingstone, Edinburgh (1985)—incorporated herein by reference in its entirety).

Stained sections were examined for necrosis, apoptosis, inflammation and vascular changes in renal tissue. The hepatic tissue was evaluated for any alterations in the architecture, portal or lobular inflammation, sinusoidal dilatation and congestion along with presence of granulomas, degeneration, necrosis and fatty change.

Histopathological grading for renal lesions. Renal lesions in [Au(en)Cl₂]Cl dosed rats were assessed by light microscopy and graded into five categories by utilizing a scale of 0 to 5 as mentioned and adopted by Zhang et al. (Zhang et al., “Immunolocalization of Kim-1, RPA-1, and RPA-2 in Kidney of Gentamicin-, Mercury-, or Chromium-treated Rats: Relationship to Renal Distributions of iNOS and Nitrotyrosine,” Toxicol. Pathol. 36(3): 397-409 (2008)—incorporated herein by reference in its entirety.):

0=normal histology,

1=tubular epithelial cell degeneration, without significant necrosis/apoptosis;

2−5=0.25%, 0.50%, 0.75% and 0.75% of the tubules showing tubular epithelial cell necrosis/apoptosis, respectively, accompanied by other concomitant alterations.

Histopathological categorization of hepatic lesions.

The hepatic lesions were categorized according to the criteria mentioned below by Ramchandran et al. (Ramachandran et al., “Histological patterns in drug-induced liver disease,” J. Clin. Pathol. 62: 481-92 (2009)—incorporated herein by reference in its entirety) (table 1).

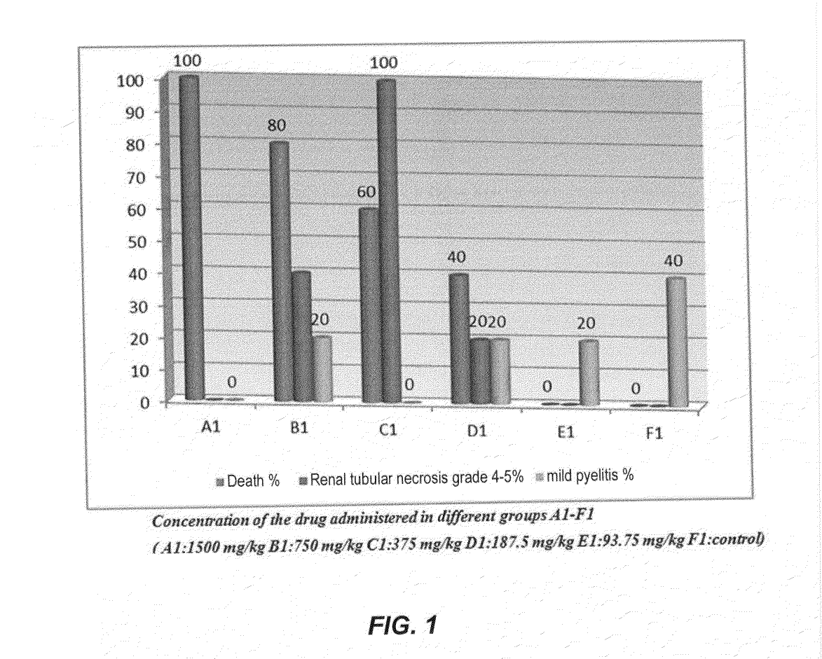

Results—The results of the study are depicted in tables 2, 3, 4 and FIGS. 1-7.

Acute Toxicity

Renal Microscopic Findings. The renal lesion in all groups of this batch demonstrated variable extent of renal tubular necrosis/apoptosis (FIG. 1) with one grade showing slight predominance over the other. No single group specific necrosis grade was evident in the entire series.

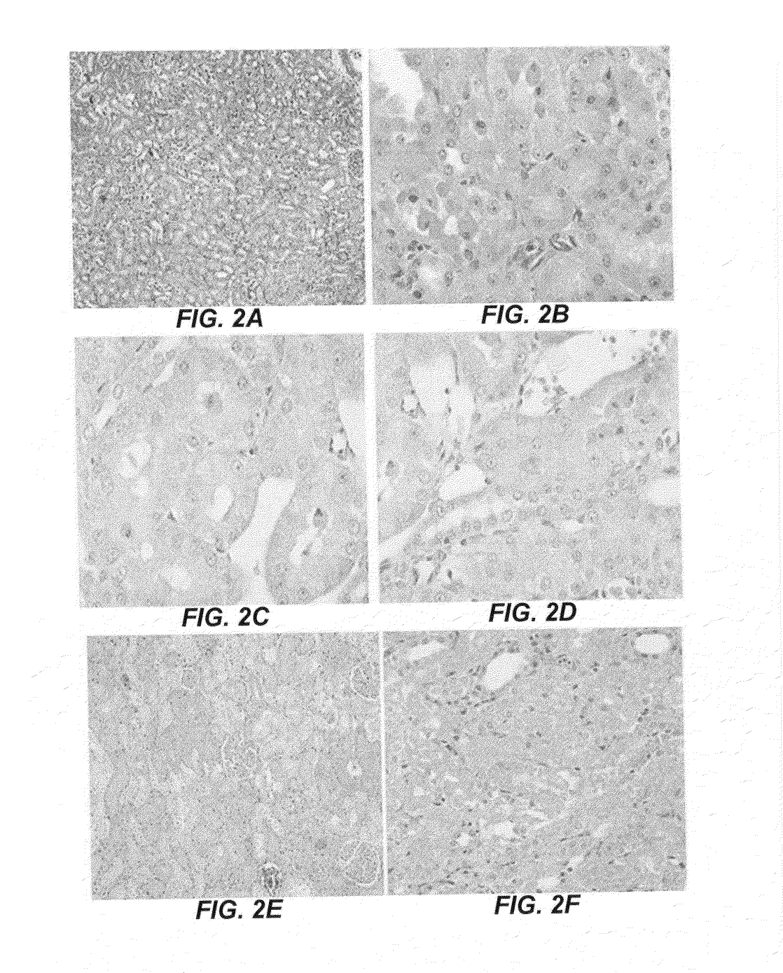

All the 5 rats in group A/I (Dose: 1500 mg/kg) died before sacrificing. The renal microscopy revealed normal histology in three animals and tubular necrosis of grade 2 severity i.e. comprising less than 25% of the total tubular tissue, in the remaining two cases (FIGS. 2a and 2b). Scattered occasional tubules with vacuolated cytoplasm were also seen along with one of the case showing cells with strongly eosinophilic cytoplasm.

In group B/I (Dose: 750 mg/kg), four out of five animals died before sacrificing. Again, a large range of necrosis was discerned, with three animals revealing grade 1 (FIGS. 2c and 2d), one grade 4 and the last grade 5 tubular necrosis.

In group C/I (Dose: 375 mg/kg), three out of five animals died before sacrificing. All animals showed renal tubular necrosis comprising 75% or more of the total renal tissue examined (grade 5, FIGS. 2e and 2f).

Group D/I (Dose: 187.5 mg/kg) had two dead animals out of five, before sacrificing. A wide range of renal tubular necrosis comprising around 25% to more than 75% of total tissue (predominantly grade 2) was discerned.

Group E/I (Dose: 93.75 mg/kg) with all 5 animals alive at necropsy, revealed renal tubular necrosis varying in range from individual cell necrosis/apoptosis to necrosis constituting less than 50% of the total renal tissue examined (predominantly grade 2-3).

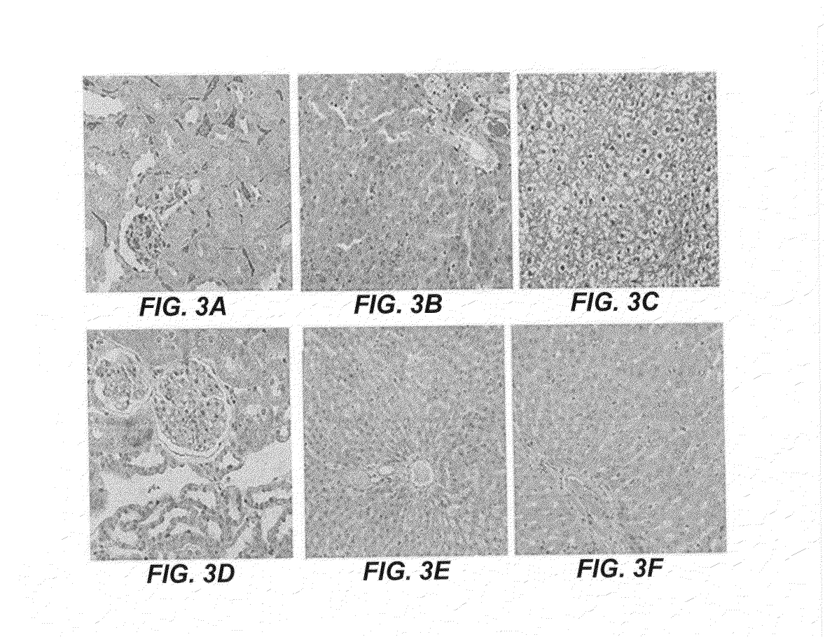

The control group (F/I) with all animals alive revealed normal renal tubular histology (FIG. 3a).

Varying extent of congestion dominated the entire histopathological spectrum.

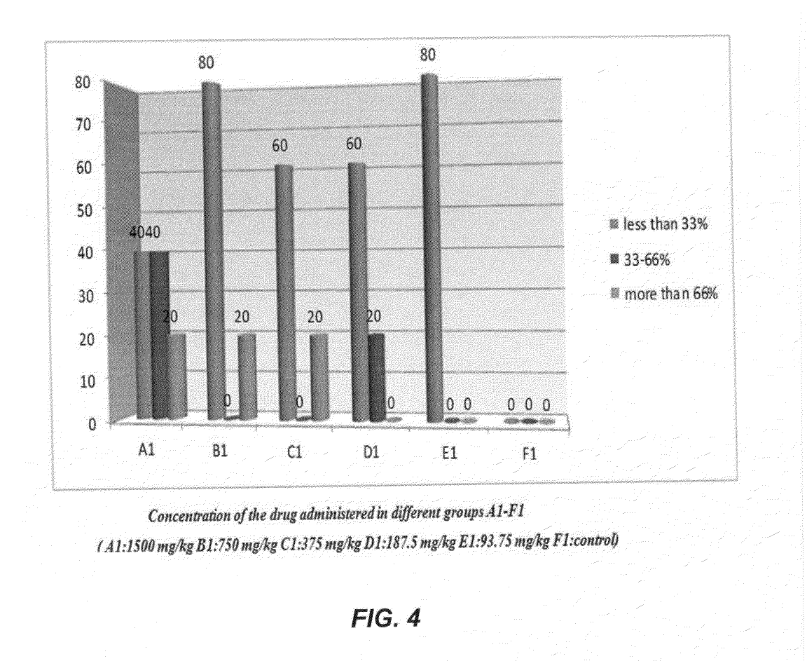

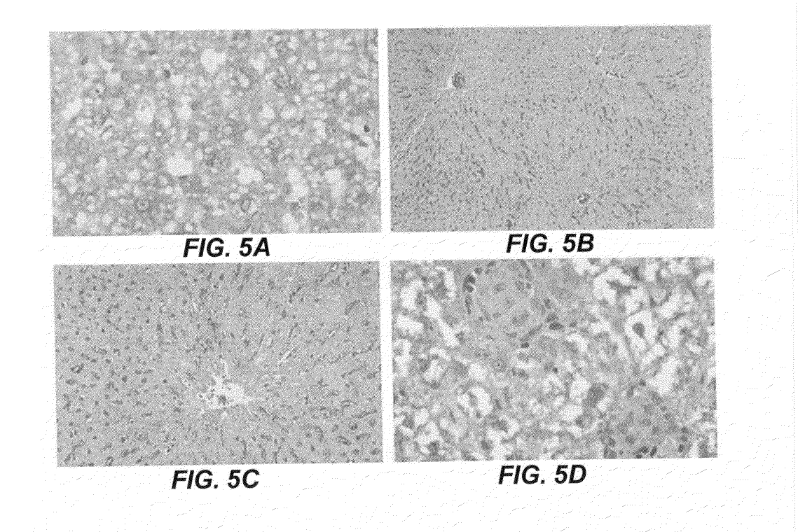

Hepatic microscopic findings. The hepatic specimens of almost all 5 animals of each group, A/I, B/I, C/I, D/I and E/I revealed variable extent of micro and macro-vesicular steatosis (FIG. 4 and FIG. 5a). Varying extent of congestion (FIGS. 5b and 5c) along with few cases showing sinusoidal obstruction syndrome were also present. In All and B/I, one and two cases respectively, revealed scattered individual hepatocytic cell degeneration without inflammation. One case showing focal necrosis with inflammation and another one revealing moderate ballooning degeneration with an occasional microgranuloma was seen in group E/I (FIG. 5d).

The hepatic picture in F/I (control, drug free group, FIGS. 3b and 3c) comprised moderate to marked ballooning degeneration (percentages of hepatic lesions are shown in table 2).

Sub-Acute Toxicity

This batch had two groups, each comprising 6 animals. The first group (A/II) was dosed with 32.2 mg/kg ( 1/10 of LD50) for two weeks and the second (group B/II) was the drug free control group.

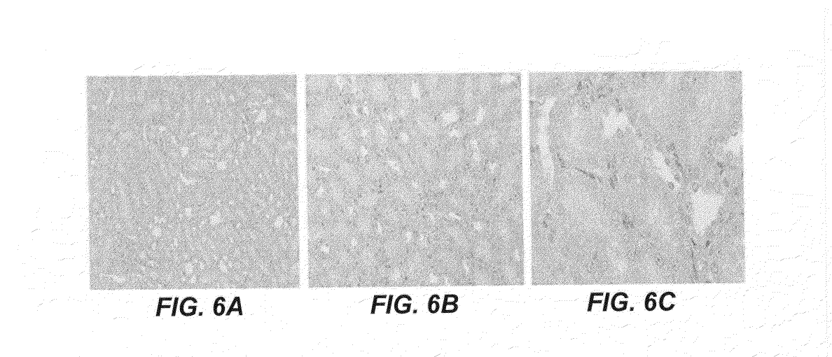

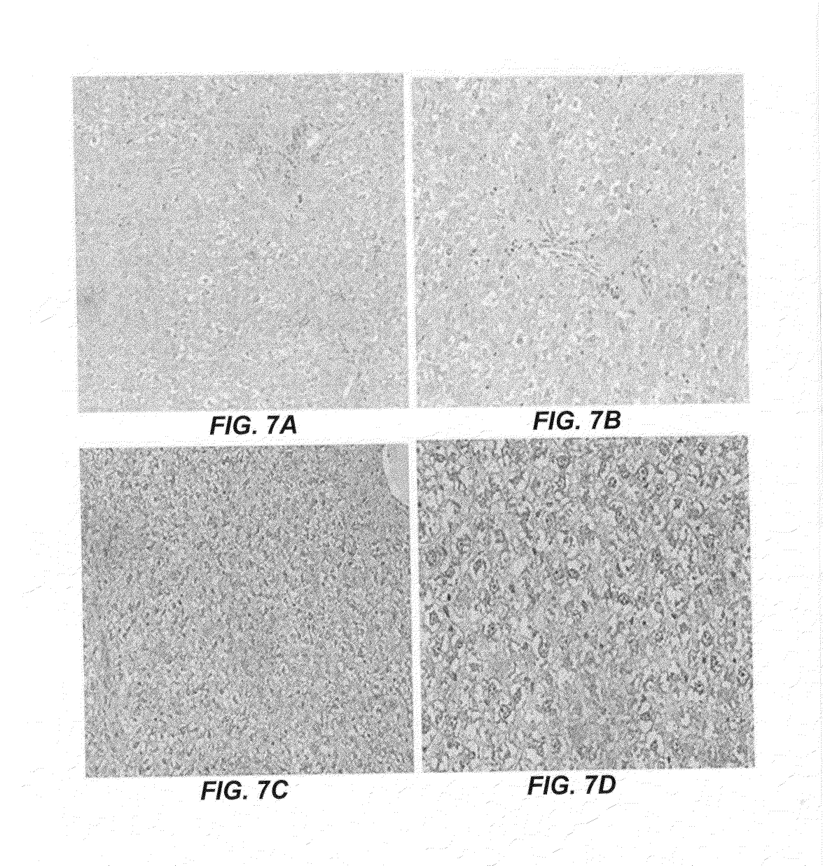

Group A/II had no animal dead before necropsy. As a whole, the renal tissue was unaffected as far as tubular necrosis (FIG. 6) was concerned. Varying extents of pyelitis with prominence of eosinophils and mild congestion spanned the entire histological picture (percentages are shown in table 3). The hepatic lesion comprised mild to marked ballooning degeneration (FIG. 7) and congestion, with one case revealing an occasional microgranuloma.

Capsular inflammation, focal portal inflammation and an occasional focus of lobular inflammation completed the entire histological spectrum (percentages are shown in table 4).

In Group B/II the renal histology was within normal limits (FIG. 3d) with pyelitis, congestion and focal pigment deposition constituting the consistent microscopic findings (table 3). The hepatic picture ranged from normal, unaffected liver (FIGS. 3e and 3f) in three cases to mild, moderate and marked ballooning degeneration, respectively, in the remaining three cases in this group (table 4). No steatosis was present in animals of this group.

The study demonstrated minimal renal and hepatic toxicity by the gold (III) compound, [Au(en)Cl₂]Cl. In the subacute toxicity part of the study, this compound showed dose dependent renal toxicity but with a much extended nephrogenic safety range and also exhibited a notably higher safe upper limit compared to toxicity levels of clinically established antineoplastic drugs like cisplatin, doxyrubicin and 5-Florouracil(5-FU) as reported in other studies. Comparative analysis with other gold compounds was limited by paucity of toxicity studies. Many studies report gold(III) complexes as emerging, potential anticancer agents (Bindoli et al., “Thioredoxin reductase: a target for gold compounds acting as potential anticancer drugs,” Coord. Chem. Rev. 253(11-12): 1692-07 (2009); Magherini et al., “Exploring the biochemical mechanisms of cytotoxic gold compounds: a proteomic study,” J. Biol. Inorg. Chem., 15(4): 573-82 (2010); Chow et al., “A gold(III) porphyrin complex with antitumor properties targets the Wnt/beta-catenin pathway,” Cancer Res. 70(1): 329-37 (2010); Yan et al., “Cyclometalated gold(III) complexes with N-heterocyclic carbene ligands as topoisomerase I poisons,” Chem. Commun. (Camb) 46(22): 3893-5 (2010)—incorporated herein by reference in their entireties) with elaboration of their mechanisms of action and antiproliferative activity against many different cancer stem lines, but their toxicity data as regards detailed renal and hepatic histopathological manifestations have not been adequately described.

The examples show a dose of 32.2 mg/kg ( 1/10 of LD₅₀) revealed normal renal tubular histology with no evidence of tubular necrosis. Mild pyelitis with a prominence of eosinophils and mild congestion was a consistent finding. Varying extent and grade of renal tubular necrosis was only seen with the administration of the gold(III) compound at very high dosages (range of 187.5-1500 mg/kg), administered in the acute toxicity component of the study.

Other antineoplastic drugs are seen to exhibit a significantly low renal tolerance. In a study comprising multi drug analysis by Hanigan et al, rats dosed intraperitoneally with 15 mg/kg of body weight cisplatin revealed grade 4 tubular necrosis (Hanigan et al., “c-Glutamyl Transpeptidase-Deficient Mice Are Resistant to the Nephrotoxic Effects of Cisplatin,” Am. J. Pathol., 159(5): 1889-94 (2010)—incorporated herein by reference in its entirety). Atasyara et al described remarkable epithelial vacuolation, necrosis, and desquamation of cells with protein casts in renal tubules after a single intraperitoneal dose of 7.5 mg/kg of cisplatin (Atasayara et al., “Preventive effect of aminoguanidine compared to vitamin E and C on cisplatin—induced nephrotoxicity in rats,” Exp. Toxicol. Pathol., 61(1): 23-32 (2009)—incorporated herein by reference in its entirety). In a study by Ravindra et al, rats injected intraperitoneally with 0.4 mg/kg of cisplatin for a period of 8 weeks showed different alterations comprising marked proximal tubular dilation and desquamation along with acute tubular necrosis (Ravindra et al., “Cisplatin induced histological changes in renal tissue of rat,” J. Cell. Animal Bio. 4(7): 108-11 (2010)—incorporated herein by reference in its entirety). Other drugs like methrotrexate and cyclosporine have been reported to have a nephrotoxic effect culminating to cell death by direct tubular toxicity and intratubular precipitation (Gronroos et al., “Methotrexate induces cell swelling and necrosis in renal tubular cells,” Pediatr. Blood Cancer 1:46(5): 624-9 (2006) and Rollino et al., “Cancer treatment-induced nephrotoxicity: BCR-Abl and VEGF inhibitors,” G. Ital. Nefrol. 50: S70-4 (2010)—incorporated herein by reference in their entireties) along with proximal tubular apoptosis and necrosis (Healy et al., “Apoptosis and necrosis: mechanisms of cell death induced by cyclosporine A in a renal proximal tubular cell line,” Kidney Int. 54(6): 1955-66 (1998)—incorporated herein by reference in its entirety) respectively, but studies evaluating their dose dependent renal histopathological manifestations are not available.

Nephrotoxicity is an integral and inherent accompaniment of multiple anti-neoplastic drugs (Yao et al., “Cisplatin Nephrotoxicity: A Review: oxygen species,” Am. J. Med. Sci., 334(2): 115-24 (2007); and Arany et al., “Cisplatin nephrotoxicity,” Semin. Nephrol. 2003; 23: 460-4 (2010); and Basu et al., “Cellular responses to Cisplatin-induced DNA damage,” J. Nucleic Acids doi:10.4061/2010/201367 (2010)—incorporated herein by reference in their entireties) which usually have a narrow therapeutic index and the minimum dosage required to significantly decrease tumor burden is usually associated with substantial nephrotoxicity. The significantly diminished renal toxicity of N-substituted ethylenediamine complexes of gold could be attributed to their different anti-proliferative mechanism of action and selective sparing of the proximal tubular epithelial cells.

Their mechanism although not precisely delineated, comprises a cumulative impact on induction of cell cycle blockage, interruption of the cell mitotic cycle, programmed cell death (apoptosis) or premature cell death (necrosis) (Isab et al., Synthesis, characterization and anti proliferative effect of [Au(en)2]Cl3 and [Au(N-propyl-en)2]Cl3 on human cancer cell lines: Spectrochimica acta Part A Molecular and biomolecular spectroscopy 79(5): 1196-1201 (2011)—incorporated herein by reference in its entirety).

Hepatotoxicity is an entity not as extensively explored as nephrotoxicity as it does not manifest itself as a dose limiting factor (Avci et al., “Cisplatin Causes Oxidation in Rat Liver Tissues:Possible Protective Effects of Antioxidant Food Supplementation,” Turk. J. Med. Sci. 38 (2): 117-120 (2008)—incorporated herein by reference in its entirety). With our ethylenediamine derivative of gold, in the acute toxicity component of the study, varying extent of steatosis was the main finding. In the sub acute toxicity component, varying extent of ballooning degeneration with accompanying congestion and focal portal inflammation comprised the predominant histopathological lesion. One of the samples revealed an occasional focus of lobular inflammation. Capsular inflammation was also a consistent finding. Other drugs like cisplatin produce hepatoxicity in high doses (Liu et al., “Metallothionein (MT)-null mice are sensitive to cisplatin-induced hepatotoxicity,” Toxicol. Appl. Pharmacol., 149: 24-31 (1998); Martins et al., “Cisplatin induces mitochondrial oxidative stress with resultant energetic metabolism impairment, membrane rigidification and apoptosis in rat liver,” J. Appl. Toxicol., 28(3): 337-44 (2008)—incorporated herein by reference in their entireties). El-Sayyad et al investigated the effects of cisplatin, doxorubicin and 5-FU belonging to different chemical classes on rats liver and showed that groups receiving cisplatin and doxorubicin exhibited increased hepatoxicity in comparison to 5-FU treatment. The most pronounced histopathlogical abnormalities observed were hepatic cord dissolution (El-Sayyad et al., “Histopathological effects of cisplatin, doxorubicin and 5-flurouracil (5-FU) on the liver of male albino rats,” Int. J. Biol. Sci., 28; 5(5): 466-73 (2009)—incorporated herein by reference in its entirety). Avci et al demonstrated that a dose of 10 mg/kg cisplatin could induce sinusoidal congestion, hydropic and vacuolar degeneration, extensive disorganization in hepatocytes, and significant fibrosis around central venules and expanded periportal areas. In another multidrug, multimodal study by Kart et al, moderate to severe hydropic degeneration in centrilobular zones extending towards the portal region was obtained with a single intraperitoneal 6.5 mg/kg dose of cisplatin. Necrotic hepatocytes, especially concentrated around the central veins, were observed in the severely affected cases (Kart et al., “Caffeic acid phenethyl ester (CAPE) ameliorates cisplatin-induced hepatotoxicity in rabbit,” Exp. Toxicol. Pathol., 62(1): 45-52 (2010)—incorporated herein by reference in its entirety).

Ballooning degeneration was a finding that was also evident in the control group of animals as well. As regards ballooning degeneration, the non significant difference between controls and drug dosed rats in hepatic toxicity in the sub-acute group reflects that drug toxicity may not be the only reason for the hepatic lesion.

The hepatic lesion produced by N-substituted ethylenediamine complexes with gold was substantially milder than cisplatin with no evidence of apoptosis or necrosis in the entire series of animals receiving a drug dose of 32.2 mg/kg for 14 days.

The gold (III) compound [Au(en)Cl₂]Cl in sub-acute toxicity study, produced less renal and hepatic toxicity as compared to other clinically established antineoplastic drugs. In the entire series of animals, no renal tubular necrosis was seen. Mild pyelitis and congestion dominated the histopathological picture. In hepatic tissue, ballooning degeneration of varied extent and severity prevailed in the drug dosed animals with no evidence of hepatocytic degeneration and necrosis.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, define, in part, the scope of the foregoing claim terminology such that no inventive subject matter is dedicated to the public.

1-2. (canceled)

3. A method of treating a patient in need of treatment for breast, ovarian, prostate or gastric cancer, comprising:
administering an effective amount of an antineoplastic drug to the patient such that no renal tubular necrosis occurs in the patient;
wherein the antineoplastic drug comprises a monomeric gold (III) compound and one or more selected from the group consisting of a pharmaceutically acceptable diluent and a pharmaceutically acceptable adjuvant, the monomeric gold(III) compound having the formula [Au(en)Cl₂]Cl where (en) is ethylenediamine.

4. (canceled)

5. The method of claim 3, wherein the antineoplastic drug is administered in an amount to produce less hepatic toxicity in comparison to the administration of a cisplatin drug.

6. The method of claim 3, wherein the antineoplastic drug is administered in a dose of 32.2 mg/kg or less.

7. The method of claim 6, wherein the antineoplastic drug is administered in a dose of 32.3 mg/kg or less per day for 14 days.

8. The method of claim 3, wherein the antineoplastic drug is administered in an amount to produce no hepatocytic degeneration.

9. The method of claim 3, wherein the antineoplastic drug is administered to the patient by injection of a saline solution comprising the antineoplastic drug.

10. The method of claim 3, wherein the patient is in need of treatment for breast cancer.

11. A method of inhibiting cancer cell proliferation in a mammalian subject having cancer, comprising:
administering to the mammalian subject, an effective amount of a gold(III) compound having the formula [Au(en)Cl₂]Cl where (en) is ethylenediamine, such that no renal toxicity occurs in the mammalian subject;
wherein the cancer is selected from the group consisting of breast, ovarian, prostate and gastric cancers.

12. The method of claim 11, wherein the safe and effective amount is 32.2 mg or less per kg of the subject body weight per day.

13. The method of claim 12, wherein the safe and effective amount is 32.2 mg or less per kg of the subject body weight per day for 14 days.

14. The method of claim 11, wherein no renal tubular necrosis occurs in the mammalian subject.

15. The method of claim 11, wherein minimal hepatic toxicity occurs in the mammalian subject.

16. The method of claim 11, wherein the administering is carried out by intraperitoneal injection.
